(19) United States
(12) Patent Application Publication
(10) Pub. No.: US 2020/0129407 A1
(43) Pub. Date: Apr. 30, 2020

(54) COSMETIC AGENT FOR TEMPORARILY RESHAPING KERATINOUS FIBRES WITH FILM FORMER OF NATURAL ORIGIN

(71) Applicant: Henkel AG & Co. KGaA, Duesseldorf (DE)

(72) Inventors: MARCUS NOLL, Quickborn (DE); SANDRA BRANDT, Pinneberg (DE); NORA KOOPMANN, Hamburg (DE)

(73) Assignee: Henkel AG & Co. KGaA, Duesseldorf (DE)

(21) Appl. No.: 16/654,293

(22) Filed: Oct. 16, 2019

(30) Foreign Application Priority Data
Oct. 25, 2018 (DE) 10 2018 218 270.1

(51) Int. Cl. A61K 8/73; A61K 8/92; A61K 8/34; A61Q 5/06

(52) U.S. Cl. CPC A61K 8/732; A61K 8/922; A61Q 5/06; A61K 8/342; A61K 8/925

(57) ABSTRACT

A cosmetic agent for temporarily reshaping keratinous fibers, for example human hair, including a film former and a cosmetic carrier, wherein the film former comprises at least one natural wax with a melting point of at least about 32° C. and at least one non-ionic propylene oxide-modified starch.

### CROSS-REFERENCE TO RELATED APPLICATION

This application claims priority to German Patent Application No. 10 2018 218 270.1, filed Oct. 25, 2018, which is incorporated herein by reference in its entirety.

### TECHNICAL FIELD

The present disclosure relates to cosmetic agents for temporarily reshaping keratinous fibers, including a film former and a cosmetic carrier, and use thereof.

### BACKGROUND

Cosmetic agents that are used to permanently or temporarily shape the hair play an important role in the field of cosmetics. Temporary styles that should have good hold without detriment to the healthy appearance of the hair, for example its shine, can be attained for example by hairsprays, hair waxes, hair gels, hair mousses, hair setting lotions, etc.

The most important property of an agent for temporarily deforming keratinous fibers is that of giving the treated fibers the strongest and longest hold possible in the created form. If the keratinous fibers are human hair, reference is also made to a strong hairstyle hold or a high degree of hold of the cosmetic agent. The hairstyle hold is determined substantially by the type and amount of used polymers.

Numerous cosmetic agents are known in the prior art for ensuring a temporary styling. Corresponding agents usually contain synthetic polymers as styling components.

The synthetic polymers usually used in agents for temporary styling are produced from corresponding synthetically accessible monomers. Said monomers are recovered from fossil substances, such as petroleum, by conversion into the corresponding polymer building blocks, inter alia with a high energy consumption. Since the monomers are already produced synthetically, the polymers usually used are fully synthetic polymers. Within the scope of a conservation of raw materials and energy, it remains desirable to use, for cosmetic products, only cosmetic raw materials that are accessible from renewable raw materials with minimal energy consumption. A reduction in amount or even a complete replacement of said fully synthetic polymers can be implemented, however, only if the replacement polymers bring about the properties desired for the intended application and give the keratinous fibers a sufficient, stable hold in the styled form.

Numerous further properties that the cosmetic agent must bestow on the hair must still be maintained nevertheless with a reduction in amount or replacement of fully synthetic polymers. Cosmetic agents are thus intended to give the hair volume, and the bounce and silkiness of the keratinous fibers fixed in the form should be maintained. The formation of polymer particles visible with the naked eye on the keratinous fibers must be avoided. Furthermore, the keratinous fibers must not look dull, but instead should appear to have a natural shine. These and further advantageous properties that the cosmetic agent provides the hair with shall be referred to hereinafter as styling properties. The development of cosmetic agents that have all desired styling properties in combination still poses difficulties. In particular, this is true for the combination of a strong hold on the one hand and simple, uniform application to the keratinous fibers on the other hand.

Document EP 2490655 A1 describes cosmetic agents for the temporary reshaping of keratin fibers, which agents contain at least one starch modified with propylene oxide.

### BRIEF SUMMARY

One aspect of the present disclosure lies in providing a cosmetic agent which is suitable for temporarily reshaping keratinous fibers, in which the use of fully synthetic polymers is significantly reduced or in which the use of fully synthetic polymers is omitted, wherein the styling properties are to be maintained at a high level. In particular, the polymers giving the hair the stable hold should be substantially free from fully synthetic polymers.

A first subject of the present disclosure is therefore a cosmetic agent for temporarily reshaping keratinous fibers, in particular human hair, comprising a film former and a cosmetic carrier, wherein the film former comprises at least one natural wax with a melting point of at least about 32° C. and at least one non-ionic propylene oxide-modified starch. In an exemplary embodiment of the present disclosure the cosmetic agent is substantially free from fully synthetic film former.

As contemplated herein the cosmetic agent comprises a film former. A film former is understood to be constituted by polymers that, as they dry, leave behind a continuous film on the skin, the hair or the nails. Film formers of this kind can be used in a wide range of different cosmetic products, such as face masks, make-up, hair fixing agents, hairsprays, hair gels, hair waxes, hair conditioners, shampoos or nail varnishes. Polymers which have sufficient solubility in water, alcohol or water/alcohol mixtures are suitable. Corresponding solutions which can be applied or further processed in a simple way can thus be produced.

Within the scope of the present disclosure a film former is understood to be constituted in particular by polymers which, when applied in from about 0.05 to about 20% by weight (in relation to the total weight of the cosmetic agent) aqueous, alcoholic or aqueous-alcoholic solution are able to deposit on the hair in the form of a transparent polymer film.

Within the scope of the present disclosure it is desirable that the film former comprises two components. A first component is a natural wax. A second component is a non-ionic propylene oxide-modified starch. Both polymers are constituents based on renewable raw materials which are processed so as to be suitable for use as film formers in the cosmetic agent.

The cosmetic agent of the present disclosure should be substantially free from polymers which have been produced fully synthetically acting as film formers. Within the scope of the present disclosure a film former is understood to have been produced fully synthetically if all monomers constructing the film former are conventionally produced synthetically. Examples of fully synthetically produced film formers comprise in particular copolymers based on acrylate or methacrylate, in particular those that comprise, as monomer constituent, acrylic acid, methacrylic acid, alkyl(meth)acrylate, hydroxyalkyl(meth)acrylate and/or maleic acid anhydride; and copolymers which comprise, as monomer constituents, N-vinylpyrrolidone, alkyl vinyl ethers with alkyl groups comprising from 1 to 18 carbon atoms and/or vinyl esters of carboxylic acids with from 2 to 18 carbon atoms, in particular polyvinylpyrrolidone/vinyl acetate copolymers; and also polyvinylpyrrolidone, wherein copolymers are understood to mean polymers that include two or more different monomers, and wherein all alkyl groups of the aforesaid monomers can also be branched alkyl groups and/or alkyl groups substituted with functional groups.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has surprisingly been found that a cosmetic agent which is substantially free of fully synthetic film formers can be formulated without necessarily having to accept any losses in respect of the styling properties. Within the scope of the present disclosure, the cosmetic agent can comprise up to about-1% by weight of fully synthetic polymers which are film formers in the conventional sense, so as to be “substantially” free of fully synthetic film formers. The cosmetic agent can for example also be free of fully synthetic film formers, i.e. the cosmetic agent comprises 0-% by weight of fully synthetic film formers.

The film former of the cosmetic agent should for example consist “substantially” of the two above components. Within the scope of the present disclosure this can for example also mean that the film former of the cosmetic agent includes the two above components exclusively, i.e. to an extent of about 100-% by weight in relation to the total weight of the film former. The feature in accordance with which the film former of the cosmetic agent consists “substantially” of the two components shall be understood by a person skilled in the art such that further components can be present in the cosmetic agent in a small amount, for example up to about 1-% by weight, for example up to about 0.8-% by weight, and for example up to about 0.6-% by weight, in relation to the total weight of the cosmetic agent and can be part of the polymer film, but without being film formers in the conventional sense. In other words, the film formed from the film former can also contain further constituents.

Within the scope of the present disclosure, “keratinous fibers” shall be understood to mean fur, wool, feathers and hair, in particular human hair.

The cosmetic agent contains as component (a) at least one natural or synthetic wax which has a melting point of at least about 37° C. The cosmetic agent contains the at least one wax for example in a total amount of from about 1 to about 30% by weight, for example from about 2 to about 50% by weight, and such as from about 3 to about 10% by weight, in relation to the total weight of the cosmetic agent.

The following can be used as natural waxes: candelilla wax, carnauba wax, esparto grass wax, Japan wax, cork wax, sugar cane wax, ouricury wax, montan wax, sunflower wax, Shea Butter (INCI: Butyrospermum Parkii (Shea) Butter), fruit waxes, and animal waxes such as beeswax and other insect waxes, spermaceti wax, wool wax, and uropygial fat. It can be advantageous to use hydrogenated or hardened waxes.

The triglycerides of saturated and optionally hydroxylated C16-30 fatty acids are also suitable, for example hardened triglyceride fats (hydrogenated palm oil, hydrogenated coconut oil, hydrogenated castor oil), glyceryltribehenate or glyceryltri-12-hydroxystearate.

The wax components can also be selected from the group of esters from saturated, unbranched alkanoic acids with a chain length of from 14 to 44 C atoms and saturated, unbranched alcohols with a chain length of from 14 to 44 C atoms, provided the wax component or the totality of wax components are solid at room temperature. The wax components can be selected for example from the group of C16-36 alkyl stearates, C10-40 alkyl stearates, C2-40 alkylisostearates, C20-40 dialkylesters of dimeric acids, C18-38 alkylhydroxystearoylstearates, C20-40 alkylerucates, wherein C30-50 alkyl beeswax and cetearyl behenate also can be used.

The natural waxes can be used alone or in combination.

The second component from the group of film formers is a non-ionic propylene oxide-modified starch. Starch is a reserve carbohydrate which is stored by plants in the form of starch grains (granules) usually from about 1 to about 200 μm in size in various plant parts, for example in tubers or roots, cereal seeds, fruits, and in the pith. A usable starch non-ionic propylene oxide-modified starch can derive from starch of potatoes, corn, rice, peas, acorns, chestnuts, barley, wheat, bananas, sago, millet, sorghum, oats, barley, rye, beans, yams, arrowroot or cassava. Advantageous effects are achieved with non-ionic propylene oxide-modified tapioca starch, or with non-ionic propylene oxide-modified potato starch, or with mixtures of the two aforesaid starches. The cosmetic agent for example contains at least one non-ionic propylene oxide-modified potato starch.

Starch belongs to the homoglycan family and is a polycondensation product of D-glucose. Starch is made up of three structurally different polymers of d-glucopyranose, namely amylose, amylopectin, and what is known as an intermediate fraction. Higher plants contain 0 to about 45% by weight amylose, based on the dry substance.

The intermediate fraction, also referred to as “anomalous amylopectin,” is structurally intermediate between amylose and amylopectin. The quantitative indications defined in the context of this application for amylopectin include the intermediate fraction.

For example, the non-ionic propylene oxide-modified starch has an amylose content of less than about 25% by weight, particularly less than about 20% by weight, in each case in relation to the weight of the modified starch. It has been found that starch containing from about 17 to about 22% by weight amylose and from about 78 to about 83% by weight amylopectin is particularly suitable for achieving the styling properties.

Amylose is made up of predominantly linear α-1,4-glycosidically linked D-glucose, from about MW 50,000 to about 150,000. The resulting chains form double helices in the starch. In addition to the α-1,4 links described for amylose, amylopectin also contains α-1,6 bonds (in an amount of from about 4 to about 6%) as branching points. The average spacing between the branching points is equal to approximately 12 to about 17 glucose units. The molar mass of from about 10′ to about 7×10⁸ corresponds to approximately 10⁵ glucose units, making amylopectin one of the largest biopolymers. Said branching points are distributed over the molecule so that a bundle structure with relatively short side chains develops. Each double helix is formed by two of these side chains. As a result of the many branching points, amylopectin is relatively easily soluble in water.

A “non-ionic propylene oxide-modified starch” as presented above is understood to be a reaction product of a starch with propylene oxide. A reaction product of this kind for example includes at least one structural unit of formula (PS),

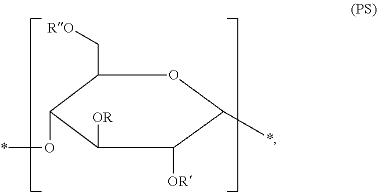

wherein at least one group R, R′, or R″ is a group of the formula

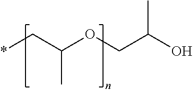

with n≤2, and at most two of the groups from R, R′, and R″ are a hydrogen atom. In formulae of this application, a bond labelled with the symbol “*” corresponds to a free valence of the corresponding structural unit. The non-ionic propylene oxide-modified starches are provided, for example, by reacting a natural starch with propylene oxide. Before modification with propylene oxide, the starch can have been exposed to a variety of physical or chemical processes, for example heat treatment, shear, thermal, acid-hydrolytic, oxidative or enzymatic cleavage, etc.

It is suitable if the non-ionic propylene oxide-modified starch is not present in the cosmetic agent in the form of individual starch grains (granules). For this purpose, starch grains are disintegrated, for example by heat or shear, and the corresponding polysaccharide molecules are released from the composite material. The released polysaccharide molecules are modified with propylene oxide after or before release.

In an exemplary embodiment, the non-ionic propylene oxide-modified starch is gelatinised. When an aqueous suspension of starch is heated or compressed, a tangential swelling of the bodies is then observed at a critical temperature or pressure, with loss of birefringence, a change in X-ray structure, and an abrupt rise in the viscosity of the solution. This phenomenon is called “gelatinisation”.

The non-ionic propylene oxide-modified starches are present in the cosmetic agent in a molecular weight distribution. Preferred non-ionic propylene oxide-modified starches have a mean molecular weight of from about 50 to about 2500 kDa (weight average). The molecular weight distribution was determined experimentally by gel filtration chromatography against dextran.

Suitable cosmetic agents contain non-ionic propylene oxide-modified starches which have a mean molecular weight (weight average) of from about 100 to about 2,000 kDa, in particular from about 500 to about 1,800 kDa, for example from about 700 to about 1,000 kDa.

In order to adjust the molecular weight, the starch is subjected to a mechanical and/or chemical treatment before or after modification with propylene oxide. To elevate the molecular weight, the starch can be crosslinked. Crosslinking of the non-ionic propylene oxide-modified starch exists when the linear or branched polysaccharide macromolecules of the starch are linked covalently by a crosslinking agent, forming a three-dimensional, insoluble, and still swellable polymeric network. Natural starch is generally considered uncrosslinked and, if crosslinking were desirable, requires artificial crosslinking by employing synthesis chemistry. Artificial crosslinking of this kind can be carried out using crosslinking agents. Non-ionic propylene oxide-modified starches that do not exhibit such crosslinking are uncrosslinked.

Crosslinking occurs, for example, by use of the crosslinking agent epichlorohydrin. For this, about a 42% by weight mixture of non-ionic propylene oxide-modified starch in water is produced, into which mixture the desired quantity of epichlorohydrin is stirred at room temperature, and, once the target viscosity is reached after a stirring time of from about 1 to about 5 hours with viscosity monitoring, the crosslinked starch is isolated using routine methods. It is suitable, however, as contemplated herein if the cosmetic agent contains at least one uncrosslinked non-ionic propylene oxide-modified starch as non-ionic propylene oxide-modified starch.

To achieve a lower molecular weight from about 100 to about 400 kDa, said starches are for example exposed to a mechanical cleavage, enzymatic cleavage (particularly using alpha-amylase, beta-amylase, glucoamylase, or debranching enzymes), acid-hydrolytic cleavage (particularly using hydrochloric acid, sulfuric acid, or phosphoric acid), thermal cleavage, or a treatment with oxidising agents (such as periodate, hypochlorite, chromic acid, permanganate, nitrogen dioxide, hydrogen peroxide, or organic percarboxylic acid, for example with hydrogen peroxide). Kneaders, extruders, stator/rotor mechanisms, and/or agitators are suitable for mechanical cleavage of the starch.

It is suitable in particular that the obtained non-ionic propylene oxide-modified starch is subjected to an oxidation treatment, for example with sodium hypochlorite or H₂O₂. For this purpose, for example the non-ionic propylene oxide-modified starch is added to water, heated to from about 50 to about 70° C., oxidant is added, and stirring is performed at from about 70 to about 85° C. for from about 2 to about 5 hours. Such starches are also referred to as 2-hydroxypropyl starch ethers.

In a suitable embodiment the film former comprises an oxidised 2-hydroxypropyl potato starch ether

The content of propylene oxide in the starch affects the fine-tuning of the hairstyle hold and hairstyle flexibility, as well as stability of the cosmetic agents. The parameters can be further optimised if the non-ionic propylene oxide-modified starch has, based on the weight of the modified starch, a propylene oxide content of from about 1 to about 20% by weight, particularly for example a propylene oxide content of from about 4 to about 12% by weight, for example a propylene oxide content of from about 9.5 to about 10.5% by weight. The propylene oxide content can be determined, for example, by carrying out a Hodges cleavage using the method according to DIN EN 13268 (date of issue June 2007).

It has also been found that those cosmetic agents in which the non-ionic propylene oxide-modified starch has, in about a 43% by weight aqueous solution, a preferred viscosity in the range of from about 150 to about 1,500,000 mPa·s (Brookfield viscosimeter, spindle 7 at about 20° C. and about 20 rpm) are outstandingly suitable for purposes of the present disclosure. Particularly suitable non-ionic propylene oxide-modified starches have viscosities from about 10,000 to about 200,000 mPa·s, particularly for example from about 25,000 to about 1800,000 mPa·s (measured in each case under the conditions described above).

It is suitable if the cosmetic agent contains the non-ionic propylene oxide-modified starch in an amount of from about 0.5% by weight to about 20% by weight, for example from about 1% by weight to about 12% by weight, more for example from about 1% by weight to about 7% by weight, and most for example from about 3% by weight to about 6.5% by weight, in each case in relation to the total weight of the cosmetic agent.

In accordance with a further preferred embodiment the cosmetic agent additionally contains an emulsifier. Emulsifiers act at the phase boundary to form water-stable and/or oil-stable adsorption layers which prevent the dispersed droplets from coalescing and thus stabilise the emulsion. Therefore, like surfactants, emulsifiers have a hydrophobic molecular component and a hydrophilic molecular component. Hydrophilic emulsifiers preferentially form O/W emulsions (oil-in-water emulsions), while hydrophobic emulsifiers preferentially form W/O emulsions (water-in-oil emulsions). Suitable emulsifiers are alkoxylated fatty alcohols. The suitable emulsifiers offer the advantage that they can be recovered from natural, non-fully synthetic raw material sources.

Accordingly, the cosmetic agent in an exemplary embodiment also contains at least one alkoxylated fatty alcohol.

An “alkoxylated fatly alcohol” is any fatty alcohol with a carbon chain of C5 or more, which also comprises at least one alkoxy group. The fatty alcohol can be selected for example from C₉-C₁₁ fatty alcohols, C₁₂-C₁₃ fatty alcohols, C₁₂-C₁₆ fatty alcohols, C₁₂-C₁₆ fatty alcohols, C₁₄-C₁₅ fatty alcohols, arachidyl alcohol, behenyl alcohol, capryl alcohol, cetearyl alcohol, cetyl alcohol, coconut alcohol, decyl alcohol, (hydrated) tallow alcohol, laurel alcohol, myristyl alcohol, oleyl alcohol, palm alcohol, palm kernel alcohol, stearyl alcohol and tridecyl alcohol.

The alkoxy group can comprise in particular ethoxy and/or propoxy and/or butoxy groups. The alkoxylated fatty alcohol is particularly for example an ethoxylated. fatty alcohol.

The at least one alkoxy group of the “alkoxylated fatty alcohol” can be derived for example from an alkoxylation reaction with alkylene oxide, in particular ethylene oxide and/or propylene oxide.

It is suitable that the at least one alkoxylated fatty alcohol is selected from the group including Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

The name Ceteareth-2 for example stands for a C₁₆-C₁₈ fatty alcohol with an average of about 2 ethylene oxide units per molecule.

In a suitable embodiment of the cosmetic agent the at least one alkoxylated fatty alcohol comprises Steareth-2 and/or Steareth-21. In a suitable embodiment of the cosmetic agent the at least one alkoxylated fatty alcohol comprises Steareth-21.

The amount of the at least one alkoxylated fatty alcohol is for example from about to about 15% by weight and more for example from about 2 to about 8% by weight, in each case in relation to the total amount of cosmetic agent.

The ease of application of the cosmetic agent can be further increased by the use of small amounts of one or more polyvalent alcohols. Preferred cosmetic agents contain one or more C3 to C12 alkane-1,2-diols, more for example C3 to C10 alkane-1,2-diols. The cosmetic agent particularly for example contains propane-1,2-diol and/or caprylyl glycol. These suitable diols improve the ease of application, are at the same time nourishing, and are suitable for forming a stable emulsion with the suitable emulsifier. In relation to the total weight of the cosmetic agent, in an exemplary embodiment of the present disclosure from about 0.01 to about 1% by weight, more for example from about 0.02 to about 0.8% by weight, in each case in relation to the total weight of the cosmetic agent, are contained by the alkane-1,2-diol in the cosmetic agent.

In an exemplary embodiment of the present disclosure the cosmetic agent contains at least one nourishing oil. Oils are advantageous as nourishing substances since they give the hair a silky shine and make the hair more resistant be being absorbed by the hair. Nourishing oils, however, place greater requirements on the product formulation since they have to be stably incorporated without demonstrating a disadvantageous settling or creaming over longer storage periods of product containers.

In accordance with an exemplary embodiment of the present disclosure the cosmetic agent comprises at least one nourishing oil and/or at least one nourishing constituent, for example at least one natural nourishing oil, i.e. a substance comprising a triglyceride of natural origin, wherein vegetable nourishing oil(s)/nourishing constituent(s) is/are suitable. Nourishing constituents should be non-polymeric in nature. Suitable are vegetable nourishing oils from the group of amaranth seed oil, argan oil, rice germ oil, baobab oil, manetti oil, marula seed oil, yangu seed oil, rambutan oil, buckthorn oil, monoi de tahiti, tigernut oil, Inca inchi oil, avocado oil, cottonseed oil, cupuacu butter, cashew oil, safflower oil, peanut oil, jojoba oil, chamomile oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and wheat germ oil, in particular (−)-α-bisabolol, hydrogenated jojoba oil and/or coconut oil. A further extremely preferred nourishing oil is myristic acid isopropyl ester (INCI: Isopropylmyristate). This occurs naturally for example in butter, palm kernel oil and coconut oil. The advantage of these nourishing oils or nourishing constituents lies in the fact that they originate to the greatest possible extent from natural sources and are thus energy- and resource-friendly raw materials. In accordance with an exemplary embodiment, the cosmetic agent can contain from about 0.5 to about 10% by weight, for example from about 1 to about 5% by weight of the sum of nourishing oils and nourishing constituents, in relation to the total weight.

As further optional ingredient the cosmetic agent can contain at least one thickening agent which is selected from homopolymers of acrylic acid or methacrylic acid and/or copolymers of acrylic acid or methacrylic acid with C₁-C₄ alkyl esters of acrylic acid or methacrylic acid.

Suitable homopolymers of acrylic acid are optionally crosslinked polyacrylic acids (INCI: Carbomer; trade name for example Carbopol) and polyacrylates as the partially or fully deprotonated salt forms thereof. The acid groups can be present fully or partially as sodium, potassium, ammonium, mono- or triethanol ammonium salt, For example, allyl ethers of pentaerythritol, of sucrose and of propylene glycol and ethylene glycol dimethacrylate are suitable as preferred crosslinking agents. Examples of homopolymers of methacrylic acid are, accordingly, optionally crosslinked polymethacrylic acids and polymethacrylates.

A particularly stable viscosity adjustment alongside simple production and low usage quantity are obtained with the use of polyacrylic acids.

It is suitable that the amount of thickening agent is up to about 1% by weight, for example up to about 0.8% by weight, and for example up to about 0.6% by weight, in each case in relation to the total weight of the cosmetic agent.

A further preferred embodiment of the cosmetic agent contains polyacrylic acid (Carbomer) thickener. In an even more preferred embodiment the cosmetic agent contains up to about 1% by weight, for example up to about 0.8% by weight, and for example up to about 0.6% by weight, in each case in relation to the total weight of the cosmetic agent, of polyacrylic acid (Carbomer) as thickener.

In further preferred embodiments of the present disclosure the cosmetic agent also contains preservatives, perfume and optionally further auxiliaries.

The cosmetic agent contains the above-described ingredients in a cosmetic carrier. The cosmetic carrier is for example an aqueous or aqueous-alcoholic carrier. The cosmetic agent contains, in relation to its total weight, for example at least about 5% by weight, more for example at least about 10% by weight, and particularly for example at least about 20% by weight water.

The cosmetic agents are for example formulated as creams. All packaging means known to a person skilled in the art and conventional for cosmetic agents for temporarily reshaping keratinous fibers, such as tubes, bottles or pots, can be used as a container.

suitable cosmetic agents comprise at least one of the following embodiments A) to I):

### A)

A cosmetic agent for temporarily reshaping keratinous fibers, in particular human hair, comprising a film former and a cosmetic carrier, wherein the film former comprises at least one natural wax with a melting point of at least about 32° C. and at least one non-ionic propylene oxide-modified starch.

### B)

A cosmetic agent for temporarily reshaping keratinous fibers, in particular human hair, comprising a film former and a cosmetic carrier, wherein the film former comprises at least one natural wax with a melting point of at least about 32° C. and at least one non-ionic propylene oxide-modified starch, and wherein the cosmetic agent is substantially free from a fully synthetic film former.

### C)

A cosmetic agent for temporarily reshaping keratinous fibers, in particular human hair, comprising a film former and a cosmetic carrier, wherein the film former, wherein the film former comprises

at least one natural wax with a melting point of at least about 32° C. and
at least one non-ionic propylene oxide-modified starch, wherein the cosmetic agent is substantially free from a fully synthetic film former.

### D)

A cosmetic agent for temporarily reshaping keratinous fibers, in particular human hair, comprising a film former and a cosmetic carrier, wherein the film former comprises at least one natural wax with a melting point of at least about 32° C. selected from the group including vegetable waxes, fruit waxes, animal waxes and mixtures thereof, and at least one non-ionic propylene oxide-modified, oxidised potato starch, wherein the cosmetic agent is substantially free from a fully synthetic film former.

### E)

A cosmetic agent for temporarily reshaping keratinous fibers, in particular human hair, comprising a film former and a cosmetic carrier, wherein the film former comprises at least one natural wax with a melting point of at least about 32° C. selected from the group including beeswax, Shea Butter (INCI: Butyrospermum Parkii (Shea) Butter) and mixtures thereof, and

at least one non-ionic propylene oxide-modified, oxidised potato starch, wherein the cosmetic agent is substantially free from a fully synthetic film former.

### F)

A cosmetic agent for temporarily reshaping keratinous fibers, in particular human hair, comprising a film former and a cosmetic carrier, wherein the film former comprises at least one natural wax with a melting point of at least about 32° C. selected from the group including vegetable waxes, fruit waxes, animal waxes and mixtures thereof, and at least one non-ionic propylene oxide-modified, oxidised potato starch, wherein the cosmetic agent is substantially free from a fully synthetic film former, and wherein the agent also contains at least one alkoxylated fatty alcohol.

### G)

A cosmetic agent for temporarily reshaping keratinous fibers, in particular human hair, comprising a film former and a cosmetic carrier, wherein the film former comprises at least one natural wax with a melting point of at least about 32° C. selected from the group including vegetable waxes, fruit waxes, animal waxes and mixtures thereof, and at least one non-ionic propylene oxide-modified, oxidised potato starch, wherein the cosmetic agent is substantially free from a fully synthetic film former, and wherein the agent also contains at least one alkoxylated fatty alcohol selected from the group including Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

### H)

A cosmetic agent for temporarily reshaping keratinous fibers, in particular human hair, comprising a film former and a cosmetic carrier, wherein the film former comprises at least one natural wax with a melting point of at least about 32° C. selected from the group including vegetable waxes, fruit waxes, animal waxes and mixtures thereof, and at least one non-ionic propylene oxide-modified, oxidised potato starch, wherein the cosmetic agent is substantially free from a fully synthetic film former, and wherein the agent also contains Steareth-21 as at least one alkoxylated fatty alcohol.

### I)

A cosmetic agent for temporarily reshaping keratinous fibers, in particular human hair, comprising a film former and a cosmetic carrier, wherein the film former comprises

at least one natural wax with a melting point of at least about 32° C., including a mixture of beeswax, Shea Butter (INCI: Butyrospermum Parkii (Shea) Butter) and
at least one non-ionic propylene oxide-modified, oxidised potato starch, wherein the cosmetic agent is substantially free from a fully synthetic film former, and wherein the agent also contains Steareth-21 as at least one alkoxylated fatty alcohol.

A second subject of the present disclosure is the use of the cosmetic agent forming the first subject of the present disclosure for temporarily reshaping and/or fixing the form of keratinous fibers, in particular human hair.

A third subject of the present disclosure is a method for temporarily reshaping keratinous fibers, in particular human hair, exemplified in that the cosmetic agent forming the first subject of the present disclosure is applied to the keratinous fibers.

It has been found to be preferred if the keratinous fibers are not rinsed following the action of the cosmetic agent forming the first subject of the present disclosure and if said agent is left on the fibers.

Features relating to preferred embodiments of the first subject of the present disclosure which, further above, have only been described in that regard also apply similarly, of course, to the second and third subject as features of preferred embodiments.

The following example is intended to explain the subject matter of the present disclosure, without limiting it in any way.

### EXAMPLE

| | % by weight |
|---|---|
| Ingredient | (active substance) |
| Beeswax (INCI: Cera Alba (Beeswax)) | 2 |
| Shea Butter (INCI: Butyrospermum Parkii | 3 |
| (Shea) Butter) and | |
| non-ionic, oxidised, propylene | 4.5 |
| oxide-modified potato starch | |
| (INCI: Hydroxypropyl Starch) | |
| Myristic acid isopropyl ester | 4 |
| (INCI: Isopropyl Myristate) | |
| Polyacrylic acid (INCI: Carbomer) | 0.6 |
| Steareth-21 | 3 |
| Preservatives | 1 |
| NaOH | 0.19 |
| 1,2-propanediol (INCI: Propylene Glycol) | 0.16 |
| Water | to 100 |

The formulation as contemplated herein was applied by hand on test subjects in order to fix the shape of the hairstyle. The hair was provided with a natural shine and a strong, but flexible hairstyle hold. In addition, the test subjects reported a positive feel of the hair.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for temporarily reshaping keratinous fibers, comprising a film former and a cosmetic carrier, wherein the film former comprises:
at least one natural wax with a melting point of at least about 32° C. and
at least one non-ionic propylene oxide-modified starch.

2. The cosmetic agent according to claim 1, wherein the cosmetic agent is substantially free from a fully synthetic film former.

3. The cosmetic agent according to claim 1, wherein the non-ionic propylene oxide-modified starch comprises a non-ionic, propylene oxide-modified tapioca starch or a non-ionic propylene oxide-modified potato starch.

4. The cosmetic agent according to claim 1, wherein the non-ionic propylene oxide-modified starch comprises a non-ionic, propylene oxide-modified potato starch.

5. The cosmetic agent according to claim 1, wherein the non-ionic propylene oxide-modified starch, in relation to the total weight of the cosmetic agent, is included in the cosmetic agent in an amount of from about 1% by weight to about 30% by weight.

6. The cosmetic agent according to claim 1, wherein the at least one natural wax is selected from vegetable waxes, fruit waxes, animal waxes and mixtures thereof.

7. The cosmetic agent according to claim 1, wherein the agent also comprises at least one alkoxylated fatty alcohol.

8. The cosmetic agent according to claim 7, wherein the at least one alkoxylated fatty alcohol is selected from Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

9. A method for temporarily reshaping keratinous fibers, comprising the step of applying the cosmetic agent according to claim 1 to the keratinous fibers.

10. The cosmetic agent according to claim 1, wherein the non-ionic propylene oxide-modified starch, in relation to the total weight of the cosmetic agent, is included in the cosmetic agent in an amount of from about 2% by weight to about 20% by weight.

11. The cosmetic agent according to claim 1, wherein the non-ionic propylene oxide-modified starch, in relation to the total weight of the cosmetic agent, is included in the cosmetic agent in an amount of from about 3% by weight to about 20% by weight.
